(19) United States
(12) Patent Application Publication
(10) Pub. No.: US 2008/0076933 A1
(43) Pub. Date: Mar. 27, 2008

(54) PROCESS FOR MAKING ANASTROZOLE

(76) Inventors: Michal Benes, Prague (CZ); Zdenek Prosek, Prague (CZ); Jiri Hykl, Prague (CZ); Jaroslav Pis, Prague (CZ)

Correspondence Address: SYNTHON IP INC, 7130 HERITAGE VILLAGE PLAZA, STE 202, GAINESVILLE, VA, 20155, US

(21) Appl. No.: 11/859,149

(22) Filed: Sep. 21, 2007

Related U.S. Application Data
(60) Provisional application No. 60/846,709, filed on Sep. 22, 2006.

(51) Int. Cl. C07D 249/08

(52) U.S. Cl. 548/267.4

(57) ABSTRACT

A process for making anastrozole using a 1-substituted triazole can reduce formation of the undesired iso-anastrozole. A typical process is represented by 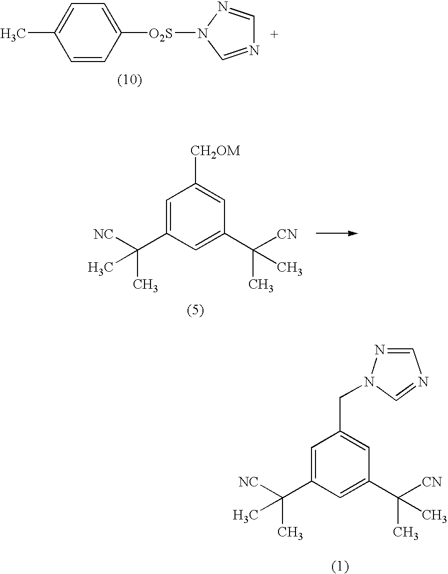 wherein compound (10) is a 1-substituted triazole and compound (1) is anastrozole.

The present invention claims the benefit of priority under 35 U.S.C. §119(e) from U.S. provisional application Ser. No. 60/846,709, filed Sep. 22, 2006, the entire contents of which are incorporated herein by reference.

### BACKGROUND OF THE INVENTION

The present invention relates to a process for making the compound anastrozole.

Anastrozole, chemically (2,2′-[5-(1H-1,2,4-triazol-1-ylmethyl)-1,3-phenylene]di(2-methylpropionitrile) of the formula (1) 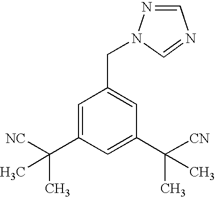
is a pharmaceutically active agent acting as a selective nonsteroidal aromatase inhibitor. Aromatase is an enzyme which regulates the level of certain female sex hormones, such as estrogens.

In pharmaceutical applications, anastrozole is used for the treatment of advanced breast cancer in post-menopausal women. In the pharmaceutical compositions it is used in the form of free base.

The whole class of 1,3-substituted aralkyl heterocyclic compounds, among which the anastrozole compound was a specific example, has been disclosed in U.S. Pat. No. 4,935,437 (reissued as US RE 36617) and the EPB 296749.

There are various processes known for the synthesis of the anastrozole. The most common ones use the 1,2,4-triazole or sodium 1,2,4-triazole as a reactant in a coupling reaction. In the original U.S. Pat. No. 4,935,437 the anastrozole compound itself was actually prepared by two variants of this process.

The first procedure (see Example 1 of EPB 296749) comprises, in the last steps, a reaction of the methyl compound of the formula (2) with N-bromosuccinimide that yields the bromomethyl-compound of the formula (3), which was treated with sodium 1,2,4-triazole to give crude anastrozole. The general pathway is shown below. 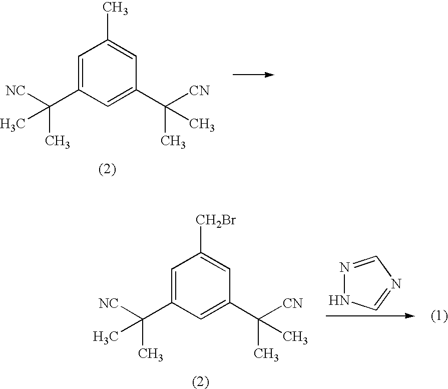
Crude anastrozole was purified by flash column chromatography (details not given) and crystallization from cyclohexane/ethyl acetate to obtain a purified material with a melting point of 81-82° C. A later document, WO 2005-105762, reported that this process was repeated with a poor yield (<50%) and poor quality (<90% by HPLC). In particular, the crystallization procedure was not successful in reducing the level of an isomeric impurity—the iso-anastrozole of the formula (4), 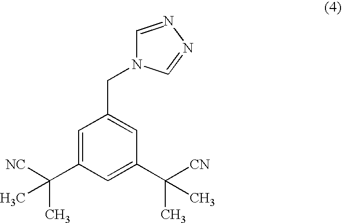
which still persisted in a level of more than 1.0% as determined by HPLC.

In a second procedure disclosed in the EPB 296749 (Example 8), the last steps use a hydroxymethyl-substituted compound, which is converted into a chloromethyl-compound of formula (6) by the reaction with thionylchloride. The crude compound (6) was reacted with 1,2,4-triazole for 18 hours at the reflux in acetonitrile to yield a mixture of anastrozole and undoubtedly the iso-anastrozole of the formula (4). After isolation the crude product was purified by flash column chromatography using methanol-chloroform mixture as the eluent. The yield and the purity of the anastrozole product were not mentioned. The general pathway is shown below.

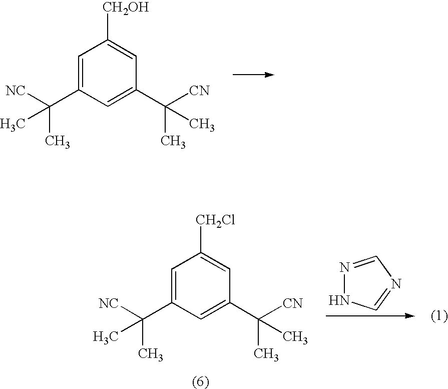

The need to use chromatographic separation in each of the above triazole-based reaction schemes makes the process economically unattractive or unviable on an industrial scale.

A different process using a substituted triazole is also disclosed in EPB 296749 (Example 69). This process comprises reacting a bromomethyl-derivative (3) with 4-amino-1H-1,2,4-triazole to give a 4-aminoanastrozolium bromide compound of the formula (7) which is then deaminated by nitrous acid (formed in situ by adding sodium nitrite to hydrochloric acid) to give anastrozole. 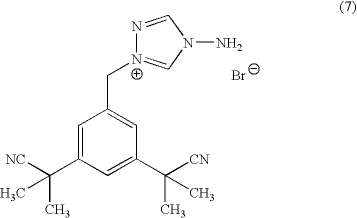
The process is believed to provide anastrozole essentially free from the iso-anastrozole impurity as the nitrogen in the 4-position is blocked. In this regard see Astleford at al. in J. Org. Chem. 1989, 54, 731-32 and more recently, Published US Patent Application US2006/0189670 which also suggests the use of 4-amino-triazole to prevent iso-anastrozole formation. But this process would appear to suffer from the use/presence of toxic and/or carcinogenic agents. Specifically, nitrosamines would be generated during the deprotecting step, which are generally difficult and/or unsuitable in making a pharmaceutical product.

Therefore, there is a need to provide an alternative production process for making anastrozole.

### SUMMARY OF THE INVENTION

The present invention relates to processes for making anastrozole using a 1-substituted triazole reactant. Accordingly, a first aspect of the invention relates to a process, which comprises: reacting a 1-substituted 1,2,4-triazole selected from the group consisting of a 1-(p-toluenesulfonyl)-1,2,4-triazole of formula (10), a sulfenyl ditriazole of formula (11), and a carbonylditriazole of formula (12) 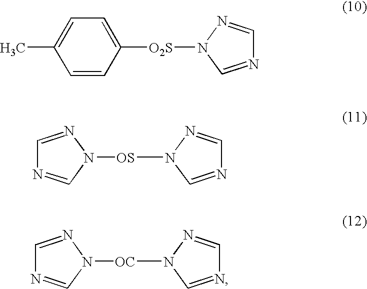
with a metal salt of 2-[3-(cyano-dimethyl-methyl)-5-hydroxymethyl-phenyl]-2-methyl-propionitrile of formula (5) 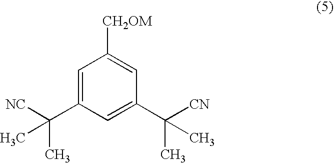
wherein M represents a metal, in a solvent to form anastrozole of formula (1) 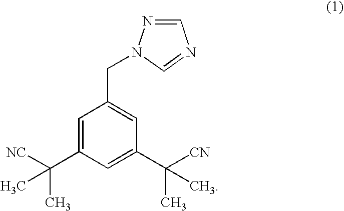
The 1-substituted 1,2,4-triazole is preferably a 1-(p-toluenesulfonyl)-1,2,4-triazole of formula (10). The metal is typically sodium and the solvent is usually a polar aprotic solvent such as dimethyl formamide, etc. The anastrozole can be isolated in solid form as the base or as a salt thereof, especially as anastrozole mesylate. The anastrozole can be purified in the salt and/or base forms.

Another aspect of the invention relates to the compounds of formula (5), especially (5a).

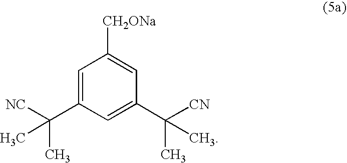

A further aspect of the invention relates to anastrozole mesylate of the formula (1a): 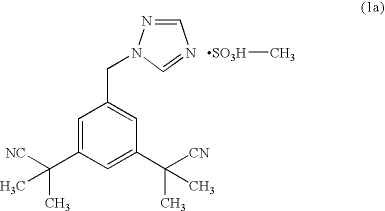
especially in solid, more preferably crystalline form. The mesylate salt is useful in isolating anastrozole and in purification.

The use of the present invention can provide anastrozole via mild reaction conditions, it can achieve good purity, and can be economically and ecologically advantageous.

### DETAILED DESCRIPTION OF THE INVENTION

In the following text, the word “triazole” will be used instead of “1,2,4-triazole” unless specifically mentioned to the contrary.

The present invention relates to the use of a 1-substituted triazole in making anastrozole. In this way, the 1-position nitrogen in the triazole compound is selectively activated for the reaction. Such selective activation could be more effective than the use of the sodium salt of triazole as taught in the art. This is because the sodium is not strictly associated to one of the nitrogen anions. As a consequence, the reaction is associated with a large amount of the isoanastrozole impurity. In contrast, the 1-tosyl triazole, having the nitrogen in the 1-position selectively activated, may be expected to have less potential in forming isoanastrozole impurities. In any event, the 1-substituted triazole can allow for the use of mild reaction conditions and is thus beneficial.

In addition to the 1-tosyl triazole of formula (10), other suitable 1-substituted reagents include sulfenylditriazole (11) and carbonylditriazole (12), 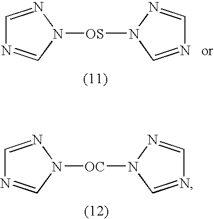
which compounds may be prepared by the reaction of triazole with thionylchloride and phosgene, respectively. The latter compound is also commercially available. However, these reagents are less preferred in the process of the invention.

The 1-substituted triazole can be used to make anastrozole via the following basic pathway, wherein 1-tosyltriazole is used as the reaction partner: 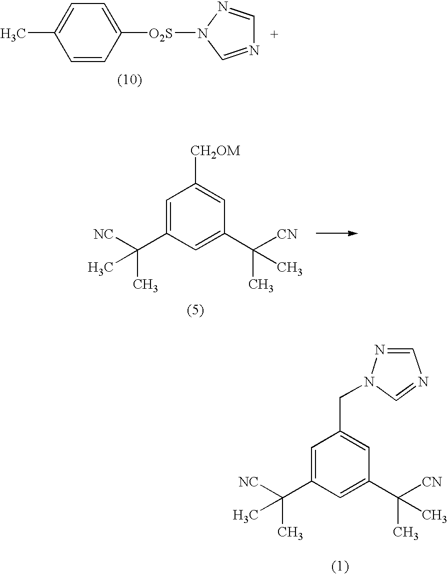
M is a metal, typically an alkali metal, and especially sodium.

The starting tosyltriazole (10) may be prepared by the following scheme: 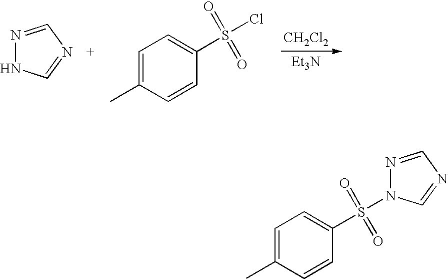
Specifically, the 1,2,4-triazole reacts with p-toluenesulfochloride in an inert solvent in the presence of a base, which is advantageously an organic base such as a tertiary amine. This base serves as a scavenger for the hydrogen chloride liberated by the reaction, but, for obvious reasons, should also be relatively inert towards the tosylation reaction. The solvent is often preferred to be an apolar or a low polar solvent, such as a hydrocarbon or a chlorinated hydrocarbon, so that the formed salt of the base can be separated as a solid and thus simply/easily removed from the reaction mixture. The solution comprising the tosyltriazole is advantageously washed with water (to remove the rest of the salts) and the product is isolated by conventional means.

The tosyltriazole can be obtained as a solid crystalline compound. It can be crystallized or recrystallized from a suitable solvent, e.g. from a hydrocarbon solvent, typically from cyclohexane. Testing of crystalline tosyltriazole revealed only one HPLC peak, which may indicate that only the 1-isomer was specifically formed.

The second reaction partner is the metal salt of 2-[3-(cyano-dimethyl-methyl)-5-hydroxymethyl-phenyl]-2-methyl-propionitrile which is represented by formula (5). The hydroxy compound (non-salt form) is known in the art and may be obtained by known methods. Prior to the reaction, it is converted into a metal salt, preferably into a sodium salt, by reaction with the metal donor, such as sodium hydride. The proper reaction partner is therefore advantageously the sodium salt (5a).

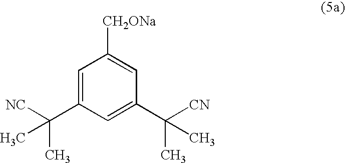

The sodium salt (5a) is a novel compound and forms a specific aspect of the present invention. The sodium salt (5a) (which may be used in an isolated form or in situ, i.e. in the solution in which it was made) reacts with the tosyltriazole (10) in a solvent yielding crude anastrozole. The expression “in a solvent” is meant in a broad sense and specifically does not require a complete solution to be formed. Thus, suspensions of solid reactants in a solvent are embraced by the expression “reacting . . . in a solvent.” Suitable solvents are advantageously polar aprotic solvents, e.g. dimethyl formamide, dimethyl acetamide, dimethyl sulfoxide, hexamethylphosphotriamide, tetrahydrofuran, dioxane, dimethoxyethane, etc. The temperature of the reaction may be ambient or lower than ambient (typically 0°-30° C.), however higher temperatures are sometimes necessary such as in solvents where the reaction proceeds in a suspension, in order to increase the level of conversion. Advantageously, the tosyltriazole is added portion wise to a suspension or solution of the sodium salt (5a) in the solvent. The reaction is slightly exothermic.

The anastrozole is usually isolated from the reaction mixture. An advantageous isolation procedure includes evaporation of the solvent and partitioning the rest between water and a water immiscible organic solvent. Water dissolves the sodium tosylate, which is the inherent by-product of the reaction, while the anastrozole concentrates in the organic phase.

Advantageously, anastrozole is isolated from the organic phase by precipitation in a form of its acid addition salt. Preferred anastrozole salt is anastrozole mesylate as it was found to nicely crystallize even from a mixture of anastrozole and side-products contained in the reaction mixture. Preferred solvent, from which the mesylate salt precipitates, is ethyl acetate.

The anastrozole mesylate of formula (1a), particularly in its isolated state and more particularly in the crystalline state, is a very advantageous intermediate in this synthetic process and forms a particular aspect of the present invention. The isolated anastrozole salt, particularly the mesylate, may be converted to the desired anastrozole base by conventional neutralization. A suitable solvent for the neutralization reaction is water.

In an alternate mode, the anastrozole base can be isolated from the organic phase by evaporating off the solvent and other volatiles.

The isolated anastrozole (either the base or the salt) may still contain some amount of the unwanted isoanastrozole and/or various other side-products. If desired, a suitable purification method may be employed. Various purification techniques are known in the art, but, the techniques explained in U.S. application Ser. No. 11/750,781, filed May 15, 2007 (the entire contents of which are incorporated herein by reference), are preferably applied to the anastrozole and/or its salts as made by the present process. In general such purification involves precipitating anastrozole from an aqueous-based solvent system such as a water/alcohol system or a dilute aqueous acid solution optionally containing alcohol. The alcohol is general a C1-C4 alcohol.

For example, anastrozole mesylate may be purified and, at the same time, converted into anastrozole base, by crystallization from a solvent comprising water. The anastrozole salts such as hydrochloride or mesylate are, in general, very sensitive towards hydrolysis in aqueous solutions, and may be hydrolysed into anastrozole base even without employing any neutralization agent (e.g. no base), although having such a neutralization agent can be convenient. Thus, a useful purification process comprises crystallization of the dissolved anastrozole salt, particularly the mesylate salt, from a solvent comprising water, particularly from a mixture of water and C1-C4 water miscible aliphatic alcohol such as methanol, ethanol or isopropanol, and optionally a neutralization agent such as an inorganic base, whereby the anastrozole crystallizes from the solvent as the free base. The crystallization of anastrozole under these conditions is accompanied with a substantive purification effect.

In another example, the crude anastrozole base is dissolved in a water miscible aliphatic alcohol, e.g. methanol or ethanol, diluted by water (using advantageously 1-2 volumes in respect to the alcohol) and optionally filtered, preferably with an activated carbon. The anastrozole-comprising solution is treated with dilute aqueous acid, especially hydrochloric acid. The amount of the acid is selected such that it preferably comprises from 0.1 to 2 molar equivalents of acid and the concentration is less than 5%, preferably less than 2%, of the total mass of the mixture. Such a dilute acid solution helps to avoid the precipitation of an anastrozole acid addition salt and instead produces the crystalline anastrozole free base, as discussed above. The aqueous solvent system containing anastrozole is generally heated to a temperature from 35° to 65° C. to achieve dissolution, etc. and cooled to a temperature not exceeding 25° C. during precipitation. In accordance with the above teaching, anastrozole precipitates as a more pure anastrozole base.

The purified anastrozole base, obtained by either of the preceding techniques or by any other technique, may be further purified by a crystallization from a mixture of water and a water miscible aliphatic alcohol, wherein the concentration of the alcohol is from about 20 to about 80% of the overall volume of the solvent mixture. The preferred alcohol is methanol and the preferred concentration is from 40 to 60%, most preferably 50% in respect to the total volume of the solvent mixture.

The above crystallization techniques can be used individually, in combination, and/or repetitively and can also be used in addition to other purification or crystallization steps. Any of the crystallization procedures can be performed by classical/traditional crystallization techniques, i.e. by heating the mixture sufficiently to insure dissolution and cooling it under precipitation of the solid. To improve the crystallization, it can be useful to inoculate the mixture with seeding crystals of anastrozole before or during cooling, and it can also be useful to dilute the mixture with water after the precipitation has started to enhance the yield.

As a result, a pharmaceutical grade anastrozole, i.e. anastrozole having the content of the title compound of at least 99.5% may be obtained in a simple and reliable process in an industrial scale.

The invention is further illustrated by the following examples, but should not be construed as being limited thereto.

### EXAMPLES

### Example 1

### 1-(p-toluenesulfonyl)-1,2,4-triazole (10)

1,2,4-triazole (9.28 g) was suspended in dichloromethane (110 mL) dried over molecular sieves. Triethylamine (13.6 g) was added; the triazole dissolved after triethylamine addition. Tosylchloride (25.62 g;) was added to the reaction mixture over approx. 30 min. The reaction mixture was stirred overnight. Precipitated salt was filtered off. Filtrate was washed with water and dried with Na₂SO₄. The drying agent was filtered off and filtrate was evaporated on rotary evaporator. Cyclohexane (300 mL) was added to the residue and the mixture was allowed to crystallise overnight. Product was separated by filtration, washed with cyclohexane (50 mL), and dried in oven at 50° C. to give the title compound as white crystalline powder, 25.1 g (83.7% of theoretical yield; m.p. 105-107° C.

### Example 2

### Synthesis of Anastrozole Mesylate

To a stirred solution of 2 g (8.25 mmol) of 2-[3-(cyano-dimethyl-methyl)-5-hydroxymethyl-phenyl]-2-methyl-propionitrile in 20 ml of dry tetrahydrofuran 0.248 g (8.25 mmol) of sodium hydride as 80% suspension in mineral oil was added. The mixture was heated to 70° C. for 30 minutes.

The mixture was then evaporated on rotary evaporator and solid residue was suspended in 10 mL of dimethylacetamide and 1.84 g (8.25 mmol) of 1-(p-toluenesulfonyl)-1,2,4-triazole was added portion-wise. The mixture was stirred 20 minutes at ambient temperature (the reaction is slightly exothermic).

Dimethylacetamide was evaporated under vacuum to leave viscous oily residue. The residue was partitioned between dichloromethane (20 mL) and water (20 mL). The layers were separated and dichloromethane layer was washed with water (20 mL). Aqueous layers were combined and washed with dichloromethane (20 mL). Combined dichloromethane layers were dried over Na₂SO₄, the drying agent was filtered off and filtrate was evaporated to give 2.58 g of oil. The oil was dissolved in ethylacetate (20 mL) and such amount of methane sulfonic acid was gradually added until liquid phase was acidic (indicator paper). Precipitated anastrozole mesylate was separated by filtration; it was washed with ethylacetate (2×5 mL) and dried at 50° C. to give 2.53 g of product (78% of theoretical yield).

### Example 3

### Synthesis of Anastrozole Mesylate

To stirred solution of 2 g (8.25 mmol) of 2-[3-(cyano-dimethyl-methyl)-5-hydroxymethyl-phenyl]-2-methyl-propionitrile in 20 ml of dry tetrahydrofuran 0.248 g (8.25 mmol) of sodium hydride as 80% suspension in mineral oil was added. The mixture was heated to 70° C. for 30 minutes.

To the solution of alcoholate cooled to 10° C. 1.84 g (8.25 mmol) of 1-(p-toluenesulfonyl)-1,2,4-triazole was added portion-wise. The mixture was stirred 20 minutes at ambient temperature, and then it was heated to 80 to 100° C. for 3 hours. The mixture was evaporated in vacuo, the remainder was partitioned between 20 ml dichloromethane and 20 ml of water. The organic extract was dried over magnesium sulfate. After filtering off the drying agent and evaporation in vacuo 2.6 g of crude anastrozole in the form of yellow oil was obtained.

The residue was dissolved in ethylacetate (20 mL) and such amount of methane sulfonic acid was gradually added until liquid phase was acidic (indicator paper). Precipitated anastrozole mesylate was separated by filtration; it was washed with ethylacetate (2×5 mL) and dried at 50° C. to give 1.79 g of product (55% of theoretical yield).

### Example 4

### Synthesis of Anastrozole Mesylate

To stirred solution of 2 g (8.25 mmol) of 2-[3-(cyano-dimethyl-methyl)-5-hydroxymethyl-phenyl]-2-methyl-propionitrile in 20 ml of dry dioxane 0.248 g (8.25 mmol) of sodium hydride as 80% suspension in mineral oil was added. The mixture was heated to 70° C. for 30 minutes.

To the solution of alcoholate cooled to 10° C. 1.84 g (8.25 mmol) of 1-(p-toluenesulfonyl)-1,2,4-triazole was added portion wise. The mixture was stirred 20 minutes at ambient temperature, and then it was heated to 80 to 100° C. for 3 hours. The mixture was evaporated in vacuo, the remainder was partitioned between 30 ml dichloromethane and 30 ml of water. The organic extract was dried over magnesium sulfate. After filtering off the drying agent and evaporation in vacuo 2.5 g of crude anastrozole in the form of a light orange oil was obtained.

The residue was dissolved in ethylacetate (20 mL) and such amount of methane sulfonic acid was gradually added until liquid phase was acidic (indicator paper). Precipitated anastrozole mesylate was separated by filtration; it was washed with ethylacetate (2×5 mL) and dried at 50° C. to give 2.09 g of product (65% of theoretical yield).

### Example 5

### Synthesis of Anastrozole Mesylate

To stirred solution of 2 g (8.25 mmol) of 2-[3-(cyano-dimethyl-methyl)-5-hydroxymethyl-phenyl]-2-methyl-propionitrile in 20 ml of dry dimethoxyethane 0.248 g (8.25 mmol) of sodium hydride as 80% suspension in mineral oil was added. The mixture was heated to 70° C. for 30 minutes.

To the solution of alcoholate cooled to 10° C. 1.84 g (8.25 mmol) of 1-(p-toluenesulfonyl)-1,2,4-triazole was added portion wise. The mixture was stirred 20 minutes at ambient temperature, and then it was heated to 80 to 100° C. for 3 hours. The mixture was evaporated in vacuo, the remainder was partitioned between 30 ml dichloromethane and 30 ml of water. The organic extract was dried over magnesium sulfate. After filtering off the drying agent and evaporation in vacuo 2.58 g of crude anastrozole in the form of a light orange oil was obtained.

The residue was dissolved in ethylacetate (20 mL) and such amount of methane sulfonic acid was gradually added until liquid phase was acidic (indicator paper). Precipitated anastrozole mesylate was separated by filtration; it was washed with ethylacetate (2×5 mL) and dried at 50° C. to give 1.02 g of product (32% of theoretical yield).

### Example 6

### Synthesis of Anastrozole Mesylate

To stirred solution of 21.71 g (89.59 mmol) of 2-[3-(cyano-dimethyl-methyl)-5-hydroxymethyl-phenyl]-2-methyl-propionitrile in 200 ml of dry dioxane 2.69 g (89.59 mmol) of sodium hydride as 80% suspension in mineral oil was added. The mixture was heated in oil bath (batch temperature) 75-85° C. for 35 minutes. The mixture was cooled down and additional amount of dioxane (100 ml) was added. To this mixture 20.00 g (89.59 mmol) of 1-(p-toluenesulfonyl)-1,2,4-triazole was added followed by additional amount of dioxane (50 ml). The mixture was gradually heated (bath temperature 115 to 125° C.) and stirred at this temperature range for total 5 hours. The mixture was then cooled down and it was left overnight. The mixture was evaporated using rotary evaporator and oily residue was partitioned between 300 ml of dichloromethane and 300 ml of water. The organic extract was dried over magnesium sulfate. After filtering off the drying agent and evaporation in vacuo 26.2 g of crude anastrozole in the form of oil was obtained.

The residue was dissolved in ethylacetate (200 mL) and such amount of methanesulfonic acid was gradually added until liquid phase was acidic (indicator paper, total amount of methanesulfonic acid was 7.2 g). Precipitated anastrozole mesylate was separated by filtration; it was washed with ethylacetate (2×50 mL) and dried at 50° C. to give 24.0 g of product (68.7% of theoretical yield).

### Example 7

### Conversion of Anastrozole Mesylate to Anastrozole

Anastrozole mesylate (24.0 g) was dissolved under heating in mixture of methanol (40 ml) and water (50 ml). Resulting solution was cooled down to 18-22° C. Water (50 ml) was added followed by aqueous ammonia solution (25%; 4.0 g). Anastrozole base started to precipitate after addition of first portions of the ammonia solution. Final pH of the mixture after addition of whole amount of ammonia solution was 1.6. Crystalline product was filtered and it was washed with mixture of methanol (15 ml) and water (35 ml). Product was dried at 50° C. to give 12.94 g of product (71.5% of theoretical yield).

### Example 8

### Recrystallization of Anastrazole Base

Anastrozole (12.94 g) containing 1.3% of 4-isomer was dissolved in a mixture of methanol (15 ml) and water (15 ml) at 40-45° C. The solution was cooled down to room temperature (product starts to crystallise) and water (15 ml) gradually added. Crystalline suspension was stirred for 30 minutes at room temperature. Crystalline product was filtered and it was washed with mixture of methanol (12 ml) and water (28 ml). Product was dried at 50° C. to give 11.7 g of product (90.4% of theoretical yield). This product was dissolved in a mixture of methanol (12 ml) and water (12 ml) at 40-45° C. The solution was cooled down to room temperature (product starts to crystallise) and water (12 ml) gradually added. Crystalline suspension was stirred for 30 minutes at room temperature. Crystalline product was filtered and it was washed with mixture of methanol (12 ml) and water (28 ml). Product was dried at 50° C. to give 11.0 g of product (94% of theoretical yield). Product contains 0.21% of 4-isomer.

Each of the patents, patent applications, and journal articles mentioned above are incorporated herein by reference in their entirety. The invention having been described it will be obvious that the same may be varied in many ways and all such modifications are contemplated as being within the scope of the invention as defined by the following claims.

We claim:

1. A process, which comprises:
reacting a 1-substituted 1,2,4-triazole selected from the group consisting of
a 1-(p-toluenesulfonyl)-1,2,4-triazole of formula (10),
a sulfenylditriazole of formula (11), and
a carbonylditriazole of formula (12) 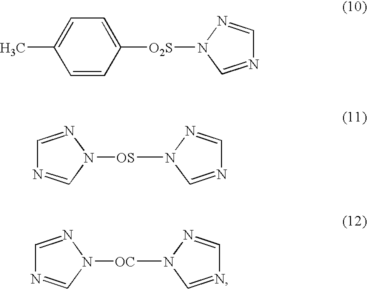
with a metal salt of 2-[3-(cyano-dimethyl-methyl)-5-hydroxymethyl-phenyl]-2-methyl-propionitrile of formula (5) 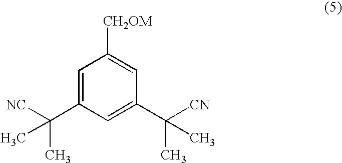
wherein M represents a metal, in a solvent to form anastrozole of formula (1)

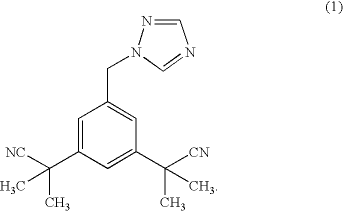

2. The process according to claim 1, wherein the 1-substituted 1,2,4-triazole is a 1-(p-toluenesulfonyl)-1,2,4-triazole of formula (10).

3. The process according to claim 2, wherein M represents sodium.

4. The process according to claim 2, wherein said reaction proceeds in a polar aprotic solvent.

5. The process according to claim 4, wherein the solvent is at least one of dimethyl formamide, dimethyl acetamide, dimethyl sulfoxide, hexamethylphosphotriamide, tetrahydrofurane, dioxane, and dimethoxyethane.

6. The process according to claim 2, which further comprises reacting said anastrozole with an acid to form an acid addition salt of anastrozole.

7. The process according to claim 6, wherein said acid addition salt is anastrozole mesylate.

8. The process according to claim 6, which further comprises precipitating said anastrozole acid addition salt.

9. The process according to claim 8, which further comprises converting said anastrozole acid addition salt into anastrozole base.

10. The process according to claim 9, wherein said converting step comprises dissolving said anastrozole acid addition salt in an aqueous solvent or solvent system and precipitating said anastrozole base therefrom.

11. The process according to claim 2, which further comprises isolating said anastrozole from the reaction mixture as a base.

12. The process according to claim 2, which further comprises purifying said anastrozole.

13. The process according to claim 12, wherein said purification comprises crystallizing said anastrozole base from a solution thereof in a diluted aqueous solution of an acid.

14. The process according to claim 12, wherein said purification comprises crystallizing said anastrozole base from a solution thereof in a mixture of water and a water miscible C1-C4 aliphatic alcohol.

15. A process of making a pharmaceutical grade anastrozole comprising the steps of:
a) reacting a compound of formula (5a) with a compound of formula (10): 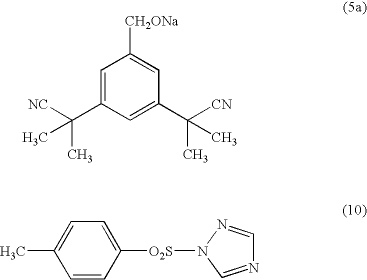
to provide anastrozole in a reaction mixture;
b) isolating anastrozole from said reaction mixture as a base or as an acid addition salt to obtain crude anastrozole;
c) dissolving said crude anastrozole in a mixture of water and a water miscible C1-C4 aliphatic alcohol, optionally in the presence of up to 2 molar equivalents of an acid, and crystallizing to form purified anastrozole; and
d) recrystallizing the purified anastrozole from a mixture of water and water miscible C1-C4 aliphatic alcohol.

16. A compound of formula (5a)

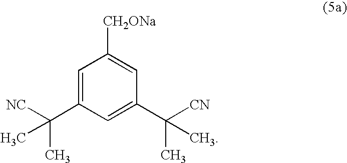

17. A compound of formula (1a)

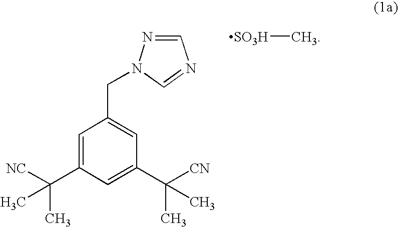

18. The compound according to claim 17 in a crystalline form.
